(19) United States
(12) Patent Application Publication
(10) Pub. No.: US 2006/0141015 A1
(43) Pub. Date: Jun. 29, 2006

(54) Antimicrobial material

(75) Inventors: Dominic Tessier, Longueuil (CA); Ion Radu, Montreal (CA); Martin Filteau, Quebec (CA)

Correspondence Address: BIRCH STEWART KOLASCH & BIRCH, PO BOX 747, FALLS CHURCH, VA, 22040-0747, US

(73) Assignee: CENTRE DES TECHNOLOGIES TEXTILES, Quebec (CA)

(21) Appl. No.: 11/295,479

(22) Filed: Dec. 7, 2005

Related U.S. Application Data
(60) Provisional application No. 60/633,458, filed on Dec. 7, 2004.

(51) Int. Cl. A61K 9/70

(52) U.S. Cl. 424/443; 442/128; 977/906

(57) ABSTRACT

The present invention relates to an antimicrobial material comprising sheet of fabric and- metallic salt crystals embedded in an adhesive material covering the sheet of fabric.

### BACKGROUND OF THE INVENTION

(a) Field of the Invention

This invention relates a new antimicrobial material having antimicrobial properties.

(b) Description of Prior Art

It is well known in the art that silver and silver salts, as well as some other metals have antimicrobial properties justifying there use in wound dressing, but also in solutions, to help healing and cicatrisation of wounds.

Antimicrobial fabrics differ in biocidal performance and durability. For example, such fabrics are used in wound dressings or bio-hazard protective clothings, and are nowadays compared in terms of “zone of inhibition” and “kill rate” of bacteria which are both related to the antibacterial activity. The material of this invention is also intended to provide a protection against biological agents (B agents) that may be used in warfare, biodefense, or counterterrorism. Among different possible B agents, are considered viruses, bacterias, and their toxins.

Other bacterial threats/diseases as the followings: bacillus anthracis/anthrax, yersinia pestisi/plague, francisella and tularensis/tularemia. Bacteria inhibition: the material in intended to stop growth. For such inhibiting material, an inhibition effect, through direct contact, can be observed and a measurable zone of inhibition may be obseved. Bacteria killing: the material, in addition of its inhibiting effect, is able to destroy microorganisms, especially bacterias.

Among the different diseases, anthrax is, for example, an acute infectious disease caused by the spore-forming bacterium bacillus anthracis. From the literature, it is suggested that antimicrobial silver can be used to inhibit and kill bacillus anthracis.

Self-decontamination technologies consist mainly of finely divided metals capable of being readily oxidized to form metal cations, metal oxides, metal hydroxides, or metal hydrates of copper, titanium, magnesium, zinc, and other metals. In addition, metals and their compounds of nanometric dimensions could also be used to provide an adsorbing surface to adsorb and bind chemical or biological materials to implement antiviral or antibiotic activity. However, nanocrystals of such metallic species may be deposited on a sorbent material having a very high surface area such as activated carbon beads or carbon cloth.

It clearly appears that a synergistic composition of different antimicrobial materials would provide the most efficient antimicrobial activity. In addition, considering that most of B agents can be disseminated as aerosol threats, surface treatment technologies, such as atmospheric plasma technology, would be advantageously applied to provide resistance to wetting by aerosol droplets containing these B agents. To achieve this, water- and oil repellent plasma coatings can be applied to fabrics and closure systems. In addition, thin plasma-deposited coatings do not alter the hand and breathability of the fabrics or membranes.

Fabrics with Silver Compounds

Antibacterial and antimicobial silver fabrics have been developed with silver compounds such as metallic siver, silver oxides, and silver salts. Silvers compounds may be extruded with a thermoplastic polymer, or dispersed in a wet-spun polymer composition, in a manner to obtain an antimicrobial fiber. Otherwise, silver compounds may be applied either as a coating in a wet process or by physical deposition technologies (see the section entitled Silver nanoparticles). Antimicrobial silver fabrics have also been prepared in the past by introducing silver fibers in the fabric structure itself.

Metallic silver, silver oxides, and silver salts are known to have antimicrobial properties; unfortunately, slow-release systems, such as metallic silver, do not confer high zone of inhibition neither high kill rate because of limited availability of silver ions in such metallic systems. Therefore, it is highly desirable to obtain antimicrobial fabrics which possess high antibacterial activity while maintaining wide-range biocidal properties.

A colloidal solution of a silver salt was applied as a coating to different fabrics. Nano-sized crystals were deposited, as observed by electron microscopy, and presented a uniform surface distribution. The silver salt nanocrystals were obtained with the use of a suitable surfactant which prevented coagulation problems and large crystal precipitation. Moreover, the use of an antimicrobial surfactant improved the antimicrobial activity of the fabric, as demonstrated by antimicrobial test methods for antibacterial activity assessment. Using different, complementary antimicrobial compounds mixed together and applied as a coating, it resulted of a greater zone of inhibition evaluated with the parallel streak test method AATCC 147, while the biocidal fabric maintained a high level of performance after commercial washing or autoclaving.

In US2003176827-2003 and WO03053484-2003, Nobel Fiber Technologies (US) provides a hydrophilic textile matrix having antibiotic activity, more precisely an antibiotic textile materials suitable for wound dressings. The textile matrix is a non-woven material including a blend (i.e., mixture) of metallic silver-coated fibers and a non-metallic, water absorbent material.

In U.S. Pat. No. 6,584,668-2003, Milliken & Company (US) discloses a method of manufacturing yarns and fabrics having a wash-durable non-electrically conductive topically applied metal-based finish. In this method, durable non-electrically conductive metal treatments (such as coatings or finishes) for yarns and textile fabrics are suggested. Such treatments preferably comprise silver and/or silver ions; however, other metals, such as zinc, iron, copper, nickel, cobalt, aluminum, gold, manganese, magnesium, and the like, may also be present or alternatively utilized. Such a treatment provides, as one example, an antimicrobial fiber and/or textile fabric which remains on the surface and does not permit electrical conductivity over the surface. The treatment is extremely durable on such substrates; after a substantial number of standard launderings and dryings, the treatment does not wear away in any appreciable amount and thus the substrate retains its antimicrobial activity (or other property). The method of adherence to the target yarn and/or fabric may be performed any number of ways, most preferably through the utilization of a binder system or through a transfer method from a donor fabric to a target textile fabric in the presence of moisture and upon exposure to heat. The particular methods of adherence, as well as the treated textile fabrics and individual fibers are also encompassed within this invention.

Also, in WO0194687-2001, Milliken discloses yarns and fabrics having a wash-durable non-electrically topically applied metal-based finish. Such treatments preferably comprise silver and/or silver ions; however, other metals, such as zinc, iron, copper, nickel, cobalt, aluminum, gold, manganese, magnesium, and the like, may also be present or alternatively utilized. Such a treatment provides, as one example, an antimicrobial fiber and/or textile fabric which remains on the surface and does not permit electrical conductivity over the surface. The treatment is extremely durable on such substrates; after a substantial number of standard launderings and dryings, the treatment does not wear away in any appreciable amount and thus the substrate retains its antimicrobial activity (or other property). Furthermore, Milliken developed an antimicrobial metal (or metal salt) coated fiber or fabric. Oeko-tex 100 certification was obtained to Milliken Chemicals for its antimicrobial compound AlphaSan, which is a silver-based inorganic additive.

In U.S. Pat. No. 6,669,966-2003, Marantech Holdings LLC (US) disclosed skin-growth-enhancing compounds and compositions including a therapeutically effective amount of at least one electron active compound, or a pharmaceutically acceptable derivative thereof, that has at least two polyvalent cations, at least one of which has a first valence state and at least one of which has a second, different valence state. Preferred compounds include Bi(III,V) oxide, Co(II,III) oxide, Cu(I,III) oxide, Fe(II,III) oxide, Mn(II,III) oxide, and Pr(III,IV) oxide, and Ag(I,III) oxide, or a combination thereof. These compounds may be in a crystalline state having metallic cations of two different valences, or electronic states, in the inorganic crystal. Also included are articles containing such compositions, such as wound dressings, and methods for facilitating or enhancing skin growth using these compounds, compositions, and articles, such as for the treatment or management of burns or skin grafts. More precisely, Marantech developed silver oxide antimicrobial textiles prepared by the deposition or interstitial precipitation of tetrasilver tetroxide (Ag4O4) crystals or its derivatives within the interstices of the fibers or yarns.

It was developed methods were silver is projected on a substrate by plasma together with an organic compound or is evaporated on the substrate together with the polymerization by plasma or an organic compound. These methods are enhancing the encapsulation of silver particles in a three-dimensional organic matrix at the surface of the substrate. However, the material obtained by these methods present discontinuities in its surface, which render the material improper for medical or high tech applications. As for example, Westaim technologies Inc has developed a product described in U.S. Pat. Nos. 5,985,308, 6,017,553, 6,080,490, 6,238,686 and 6,333,093. This product is a silver coated dressing made of three piles, the center one being made of absorbent rayon and the two external plies being covered with silver. Westaim has also developed a silver foam dressing wherein silver is incorporated in a gel made of a collagen's derivative.

Silver wound dressings were also prepared in the past by introducing silver fibers in the preparation of the dressing itself or by applying a silver salt coating to a fabric, as described by Matson in U.S. Pat. No. 4,728,323, who coated a substrate with a film of silver salt deposited by vapor or sputter coating techniques. However, this dressing is having a limited antimicrobial activity.

Argentum Medical, as described in U.S. Pat. No. 6,087,549, developped a multilayer laminate wound dressing comprising a plurality of layers of preferably silver or silver-coated fibers in a woven fabric alternating with layers of nonconductive, preferably nonmetallic, fabric. Each layer preferably contains a different ratio of metalized to nonmetalized fibers. The metalized fibers are preferably made of or coated with silver. The dressing promotes healing by stimulating cellular de-differetiation, followed by cellular proliferation. The dressing also has antibacterial, antifungal and analgesic properties. The product, registered as Silverlon® is manufactured using a metal deposition process.

Johnson & Johnson Medical developed a multilayered wound dressing as described in U.S. Pat. No. 6,348,423, U.S. Pat. No. 6,166,084, and U.S. Pat. No. 5,925,009, which includes a fibrous absorbent layer for absorbing wound exudates, an odor layer for absorbing odor and a barrier layer interposed the fibrous absorbent and barrier layers. The dressing is available as Actisorb® and comprises charcoal cloth together with silver, a silver sulfadiazine salt compound, sealed within a nylon sleeve and may further include one or more absorbent layers. Antimicrobial species are silver ions.

Silver Leaf Technologies, in the application CA2343440, describes an ultrasonic process for autocatalytic deposition of metal. The process results in the autocatalytic plating bath depositing the metal on the material in a controlled and substantially uniform thickness. The material can be selected from Nylon, Kevlar, Zylon and aramid fibers, and the metal can be silver which as effective anti-microbial properties when used in wound dressings.

Acrymed, as described in U.S. Pat. No. 6,605,751, U.S. Pat. No. 6,355,858, and U.S. Pat. No. 5,928,174, developed a silver-containing antimicrobial hydrophilic material. The stabilized silver antimicrobial devices comprise a matrix with a polymer network, a non-gelable polysaccharide, and an active agent. The product, SilvaSorb®, is composed of a matrix that may be formed into any desired shape for its desired uses, especially used in sheet or gel forms. In this particular dressing, silver chloride is an effective antimicrobial agent.

Coloplast, as described in U.S. Pat. No. 6,468,521 and U.S. Pat. No. 6,726,791, developed a stabilised composition having antibacterial, antiviral and/or antifungal activity characterised in that it comprises a silver compound and that the compound is in the form of a complex with a primary, secondary or tertiary amine which complex is associated to one or more hydrophilic polymers is stable during sterilisation and retaining the activity without giving rise to darkening or discoloration of the dressing during storage. The product, registered as Contreet® is a dressing product comprising a silver compound in the form of a complex with an amine. This silver compound is said to have improved resistance to discoloration when exposed to light or radiation sterilisation. The silver-amine complex may be used in conjunction with an hydrophilic polymer for producing a wound dressing.

ConvaTec, a Bristol-Myers Squibb Company, in U.S. Pat. No. 666,981, claims for enhancement of photostabilization of silver in medical materials. More particularly, the methods increase the photostabilization of silver in certain materials comprising hydrophilic, amphoteric and anionic polymers by subjecting the polymers to solutions containing an organic solvent and silver, during or after which one or more agents are added which facilitate the photostablization of the material. Agents comprises an ammonium salt selected from ammonium chloride, ammonium acetate, ammonium carbonate, ammonium sulphate and mixtures thereof. The polymer is subjected to the solution for a time that is sufficient to incorporate the desired silver concentration. During or after the period wherein the polymer is subjected to the solution, the polymer is subjected to one or more agents which facilitate the binding of the silver and the polymer together. Suitable agents include ammonia, ammonium salts, thiosulphates, chlorides, and/or peroxides particularly aqueous ammonium chloride. Materials which are particularly adapted for the inventive method include gel-forming fibers such as Aquacel® that can swells with the salt solution.

C. R. Bard, in U.S. Pat. No. 6,716,895, disclose polymer compositions containing colloids of silver salts. The compositions are said to advantageously provide varying release kinetics for the active ions in the compositions due to the different water solubility of the ions, allowing antimicrobial release profiles to be tailored for a given application and providing for sustained antimicrobial activity over time. More particularly, the invention relates to polymer compositions containing colloids comprised of salts of one or more oligodynamic metal, such as silver. The process of the invention includes mixing a solution of one or more oligodynamic metal salts with a polymer solution or dispersion and precipitating a colloid of the salts by addition of other salts to the solution which react with some or all of the first metal salts. The compositions can be incorporated into articles or can be employed as a coating on articles such as medical devices. However, in U.S. Pat. No. 6,716,895, no surfactant is used to stabilise the silver colloids. This method has the main disadvantage to promote rapid coagulation of the colloids.

Fabrics with Copper Compounds

Only a few fabrics with copper have been developed and commercialized for their antimicrobial proporties. These fabrics are said to possess antiviral properties, thus providing biological protection against both viruses and bacterias.

Cupron Corp (US), in DE60102291 D-2004, claims polymeric fibers, yarns, films, having an antimicrobial and antiviral ionic copper (copper salt) encapsulated within the fiber and protruding at the surface of the fiber. Cupron Corp (US), in WO0174166-2001 discloses antimicrobial and antiviral polymeric materials. The invention provides an antimicrobial and antiviral polymeric material, having microscopic particles of ionic copper encapsulated therein and protruding from surfaces thereof. In addition, in WO0075415-2000, Cupron Corp. discloses a clothing having antibacterial, antifungal, and antiyeast properties, comprising at least a panel of a metallized textile fabric, the textile fabric including fibers selected from the group consisting of natural fibers, synthetic cellulosic fibers, regenerated protein fibers, acrylic fibers, polyolefin fibers, polyurethane fibers, vinyl fibers, and blends thereof, and having a plating including an antibacterial, antifungal and antiyeast effective amount of at least one oxidant cationic species of copper.

In addition, fabrics for combating and preventing nosocomial infections (CA2407087-2001, WO0181671, and U.S. Pat. No. 6,482,424-2002) in healthcare facilities by MTC Medical Fibers (Israel), a division of Cupron Inc of NY USA, have been developed. Textiles incorporate fibers coated with an oxidant cationic form of copper and are claimed to be effective for the inactivation of antibiotic-resistant strains of bacteria, particularly methicillin-resistant Staphylococcus aureus (MRSA) and vancomycin-resistant Enterococcus (VRE).

Other Antimicrobial Fabrics with Nano-Sized Crystals

In DE10051647-2002 is disclosed a protective material in two-dimensional or three-dimensional form against chemical poisons and warfare agents comprises nano-crystals permanently fixed to the surface of a carrier element which lets through air and water vapor.

Antimicrobial Surfactants

Many surfactants may also possess antimicrobial activity and include detergent surfactant such as anionic, nonionic, zwitterionic, ampholytic and cationic surfactants.

Most popular antimicrobial surfactants are cationic surfactants which ideally comprises two long alkyl chain lengths. Examples of such cationic surfactants include the ammonium surfactants such as alkyltrimethylammonium halogenides, as detailed below in the section Quaternary Ammonium Compounds.

Quaternary Ammonium Compounds (QAC)

QAC is an antibacterial agent which may be found as many simililar compounds part the cationic surfactants category.

The Dial Corporation, in U.S. Pat. No. 6,616,922-2003, describes an antimicrobial composition including a quaternary ammonium antibacterial agent which is selected from the group consisting of cetyl trimethyl ammonium bromide, octadecyl dimethyl benzyl ammonium bromide, N-cetyl pyridinium bromide, octylphenoxyethoxy ethyl dimethyl benzyl ammonium chloride, N-(laurylcoco-aminoformylmethyl)pyridinium chloride, lauryloxyphenyl-trimethyl ammonium chloride, cetylaminophenyl trimethyl ammonium methosulfate, dodecylphenyl trimethyl ammonium methosulfate, dodecylbenzyl trimethyl ammonium chloride, chlorinated dodecylbenzyl trimethyl ammonium chloride, dioctyl dimethyl ammonium chloride, benzalkonium chloride, myristyl dimethylbenzyl ammonium chloride, methyl dodecyl xylene-bis-trimethyl ammonium chloride, benzethonium chloride, a 2-butenyl dimethyl ammonium chloride polymer, behenalkonium chloride, cetalkonium chloride, cetarylalkonium bromide, cetrimonium tosylate, cetylpyridinium chloride, lauralkonium bromide, lauralkonium chloride, lapyrium chloride, lauryl pyridinium chloride, myristalkonium chloride, olealkonium chloride, isostearyl ethyldimonium chloride, and mixtures thereof.

Other Surfactant Systems

Nonionic surfactants may be formed of polyethylene, polypropylene, and polybutylene oxide condensates of alkyl phenols. Commercially available nonionic surfactants include Igepal™. CO-630, marketed by the GAF Corporation; and Triton™. X45, X-114, X-100 and X-102, all marketed by the Rohm & Haas Company. These surfactants are commonly referred to as alkylphenol alkoxylates (e.g., alkyl phenol ethoxylates). Other commercially available nonionic surfactants include Tergitol™. 15-S-9 and Tergitol™. 24-L-6 NMW both marketed by Union Carbide Corporation; Neodol™. 45-9, Neodol™. 23-3, Neodol™. 45-7, and Neodol™. 45-5, marketed by Shell Chemical Company; Kyro™. EOB, marketed by The Procter & Gamble Company, Genapol LA O3O or O5O, marketed by Hoechst, and Tetronic™. compounds, marketed by BASF.

Anionic surfactants may be formed of linear alkyl benzene sulfonate, alkyl ester sulfonate, alkyl alkoxylated sulfate, and alkyl sulfate. Others may include salts (including, for example, sodium, potassium, ammonium, and substituted ammonium salts such as mono-, di- and triethanolamine salts) of soap.

Ampholytic surfactants can be broadly described as aliphatic derivatives of secondary or tertiary amines, or aliphatic derivatives of heterocyclic secondary and tertiary amines in which the aliphatic radical can be straight- or branched-chain.

Zwitterionic surfactants can be broadly described as derivatives of secondary and tertiary amines, derivatives of heterocyclic secondary and tertiary amines, or derivatives of quaternary ammonium, quaternary phosphonium or tertiary sulfonium compounds.

Antimicrobial Compositions

In WO9903512-1999, Procter & Gamble discloses a method for sanitizing a substrate by contacting a microbe containing substrate with a detergent composition for a sufficient time to substantially reduce the amount of microbes on the substrate. Substrates can be sanitized by applying a light duty detergent composition, preferably a liquid, cream, paste, or gel detergent composition, which comprises an antimicrobial agent such as a surfactant.

In U.S. Pat. No. 6,626,873-2003, is disclosed a polymeric coating composition comprising anti-infective agents chlorhexidine and triclosan. It is based, at least in part, on the discovery that the synergistic relationship between these compounds permits the use of relatively low levels of both agents, and on the discovery that effective antimicrobial activity may be achieved when these compounds are comprised in either hydrophilic or hydrophobic polymers. It is also based on the discovery that chlorhexidine free base and triclosan, used together, are incorporated into polymeric medical articles more efficiently. Medical articles prepared according to the invention offer the advantage of preventing or inhibiting infection while avoiding undesirably high release of anti-infective agent. In the impregnating solution, the chlorhexidine consists essentially of a mixture of chlorhexidine free base and a chlorhexidine salt.

ISP Investments Inc, in U.S. Pat. No. 6,576,230-2003, discloses a mixture of biocides designed to control unwanted microbial growth in water-based applications, including coatings, adhesives, and latex emulsions. The biocidal composition comprises a mixture of 2-propenal polymer (APC) and 5-chloro-2-methyl-4-isothiazoline-3-one (CIT) and 2-methyl-4-isothiazoline-3-one (MIT).

Stepan Company, in U.S. Pat. No. 6,492,445-2002, discloses antimicrobial polymer latexes derived from unsaturated quaternary ammonium compounds for antimicrobial coatings, sealants, adhesives and elastomers produced from such latexes. Antibacterial CASE materials comprise a latex comprising polymer particles and a surfactant component.

In U.S. Pat. No. 6,436,419-2002, is disclosed an antimicrobial treatment for polymers which consists to provides durable and refreshable antimicrobial polymeric treatments. In some instances, the polymer is a textile. These textiles are said to have excellent colorfastness and wash fastness. The antimicrobial fabrics of this invention are suitable for sportswear, antiodor carpets, films, plastics, toys and medical uses. Antimicrobial composition comprises quaternary ammonium salt attached to a dye, which is a bridge between said polymer and said antimicrobial agent and wherein said antimicrobial composition has more durable antimicrobial activity than a composition with the antimicrobial agent attached directly to the polymer thereof.

Antimicrobial fabrics, especially those used in wound dressings, are nowadays compared in terms of antibacterial activity and kill rate of living bacteria to provide useful information about efficiency of the antibacterial activity. Unfortunately, sustained, slow-release systems, such as metallic silver do not confer high antibacterial activity nor high kill rates to wound dressings because of limited availability of silver ions in such metallic systems.

It would be highly desirable to be provided with a new antimicrobial material providing a high antibacterial activity and high bacteria killing rate for wound dressings.

### SUMMARY OF THE INVENTION

In accordance with the present invention there is provided an antimicrobial material comprising
a sheet of fabric and;
metallic particles embedded in an adhesive material covering said sheet of fabric.

The metallic particles are intended to be salts, oxides or hydroxides. It is preferable to use a slightly soluble metallic salt or oxide since this allow controlled and long term release of antimicrobial ions as well as increase the durability of the antimicrobial effect.

In one embodiment of the present invention, the metallic salt crystals are micro-sized crystals.

In a preferred embodiment of the present invention, the metallic salt crystals are nano-sized crystals.

The size of the crystals range from 10 to 1000 nm, preferably from 10 to 500 nm and more preferably from 10 to 150 nm.

The fabric suitable for the present invention can be selected from, but not limited to, nylon, aramid, acetate, flax, polyolefin such as polyester, polyethylene and polypropylene, rubber, saran, spandex, vinyl, vinyon, cotton, wool, silk, rayon, glasswool, acrylic, paper, polytetrafluoroethylene, synthetic polymers, cellulosic fibers, natural fibers, synthetic or man made fibers and mixtures thereof, preferably nylon, polyester/carbon and polyester/cotton. The fabric can be in the form of fibers, membranes or any other form suitable for performing the material of the present invention.

It is also possible to use a fabric that is electrically conductive. The fabric can be rendered electrically conductive by the incorporation of electricity conductive material thereto, such electricity conductive material being such as metallic yarn, carbon yarn and a combination thereof.

In a preferred embodiment of the present invention, the metallic salt crystals are from a metal selected from the group consisting of, but not limited to, silver, platinum, gold, copper, zinc, titanium, magnesium and mixtures thereof.

In a preferred embodiment of the present invention, the metallic salt crystals are soluble or slightly soluble salts such as, but not limited to, AgBr, silver perchlorate, AgF, AgCl, AgNO₃, silver sulfate, AgI, silver alkylcarboxylate, silver sulphadiazine, silver arylsulfonate and mixtures thereof, more preferably silver chloride.

In another embodiment of the present invention, the metallic salt crystals are from, but not limited to, CuI, CuBr, CuCl, CuF, CuBr₂, CuCl₂, CuI₂, and CuF₂.

In a further embodiment of the present invention, the metallic salt crystals are from, but not limited to, AuF₃, AuCl, AuCl₃, AuBr₃ and AuI.

Metallic oxides can be used as well in the present invention. The metallic oxides can be from a metal selected from, but not limited to silver, platinum, gold, copper, zinc, titanium, magnesium and mixtures thereof. Metallic oxides such as, but not limited to, CuO, Cu₂O, Cu(OH)₂, Ag₂O, AgO, Ag(OH) and Au₂O₃ are suitable for the present invention.

Additionally, the metallic silver and/or metallic copper can be present in the material of the present invention.

In an embodiment of the present invention, the material further comprises an antimicrobial compound. This antimicrobial compound can be selected from the group consisting of, but not limited to, quaternary ammonium compounds (QAC), chlorinated organic compounds, cetrimide, iodine compounds, hexamine hippurate, dequalinium and alcohols. Chlorinated organic compounds are preferably selected from the group consisting of 2,4,4′-trichloro-2′-hydroxydiphenol ether, chlorhexidine, hexachlorophene and 5-chloro-2-phenol (2,4-dichlorophenoxy) without limitation.

In a preferred embodiment of the present invention, the adhesive is made of monomers selected from the group consisting of, but not limited to, acetate, acrylate, acrylic, acrylamide, urethane, vinyl and ester, more preferably vinyl acetate or polyurethane.

In one embodiment of the present invention, the material further comprises a layer of metal over the metallic ions embedded in adhesive material.

In another embodiment of the present invention, the material further comprises a layer of metal between the sheet of fabric and the metallic ions embedded in adhesive material.

In further embodiment of the present invention, the material further comprises a layer of metal underneath the sheet of fabric.

Preferably, the layer of metal is a layer of silver. The silver being nanocrystalline silver or silver oxide. The layer of metal is formed by plasma deposition. The plasma used for deposition is selected from the group consisting of, but not limited to, argon, argon/oxygen, argon/nitrogen, argon/nitrogen/hydrogen, krypton, krypton/nitrogen, krypton/nitrogen/hydrogen, xenon, xenon/nitrogen, xenon/nitrogen/hydrogen, helium, helium/nitrogen, helium/nitrogen/hydrogen, neon, neon/nitrogen and neon/nitrogen/hydrogen plasma, preferably argon plasma.

The material of the present invention affects gram positive, such as, but not limited to, staphylococcus aureus and bacillus anthracis and gram negative bacteria such as, but not limited to Escherichia coli, pseudomonas aeruginosa, enterococcus faecium and salmonella. Other bacteria against which the material of the present invention is effective is E. Herbicola.

The material of the present invention also affects viruses and fungi and could be used for this purpose as well.

In a preferred embodiment of the present invention, the adhesive is transparent.

In a preferred embodiment of the present invention, the adhesive is washing durable.

The adhesive can also be selected from a antimicrobial polymer. Such antimicrobial adhesive can be selected from the group consisting of, but not limited to, siloxane monomers or polymers having been functionalized by N-halamines, iodinated resin, iodinated complexes, polymeric biguanide compounds such as poly(hexamethylene biguanide) and related cationic salts derivatives, polymerized aromatic quaternary ammonium salt monomers, poly(2-propenal, 2-propenoic acid), α,β-amino acid oligomer or polymer, and poly(2-methyl-5-vinylpyridine) or poly vinylpyrrolidone treated by iodide salt.

In a preferred embodiment of the present invention, the fabric is antistatic.

The antimicrobial fabric is prepared by the application of a coating containing silver salt colloids into which the silver salt colloids are stabilized with the use of a surfactant that lowers the surface energy of the colloidal solution. The stabilized colloids remain in smaller dimensions than their unstabilized counterparts, then providing an improved surface distribution once the coating is applied to the fabric. This better distribution of the silver salt provide greater uniformity of the applied silver salt and impedes coagulation problems and large cluster precipitation in the application bath. Moreover, the chosen surfactant may have antimicrobial properties, which confer additional antimicrobial activity to the coating.

Then, the incorporation of an antimicrobial surfactant in the coating formulation will add to the overall antibacterial activity provided that gram positive and gram negative bacteria are differently affected by various antibacterial compounds such as silver salts, quaternary ammonium compounds, chlorinated organic compounds, ethoxylated alcohols, etc. The main intent for using a combination of different antimicrobial compounds is to obtain a antimicrobial fabric showing intense, immediate antimicrobial properties over a wide range of gram positive and gram negative bacteria and that these antimicrobial properties last over time by a sustained, slow-release of antimicrobials which is provided by the ionic silver contribution.

An additional advantage provided by the antimicrobial fabric developed according to this method is that the antimicrobial coated fabric may by used in dry or in humid environment as well, without staining especially in humid wound site. For example, in comparison to fabrics coated with metallic silver, the coated antimicrobial fabric having a silver chloride salt/surfactant complex will prevent the staining of the skin if humidity is present. In many cases, according to the invention, clear, transparent-like or colorless antimicrobial coatings can be obtained.

All references herein are hereby incorporated by reference.

### BRIEF DESCRIPTION OF THE DRAWINGS

FIG. 1 illustrates a cross-sectional side view of a first embodiment of the present invention in which metallic particles (14) embedded in adhesive (16) cover a sheet of fabric (12);

FIG. 2 illustrates a cross-sectional side view of a second embodiment of the present invention in which a layer of metal (18) covers the metallic particles (14) embedded in adhesive (16);

FIG. 3 illustrates a cross-sectional side view of a third embodiment of the present invention in which the layer of metal (18) is between the metallic particles (14) embedded in adhesive (16) and a sheet of fabric (12); and

FIG. 4 illustrates a cross-sectional side view of a fourth embodiment of the present invention in which the layer of metal (18) is underneath the sheet of fabric (12).

### DETAILED DESCRIPTION OF PREFERRED EMBODIMENTS OF THE PRESENT INVENTION

In accordance with the present invention, there is provided a new antimicrobial material.

A colloidal solution of a silver salt was applied as a coating to different fabrics. Nano-sized crystals were deposited, as observed by electron microscopy, and presented a uniform surface distribution. The silver salt nanocrystals were obtained with the use of a suitable surfactant which prevented coagulation problems and large crystal precipitation. Moreover, the use of an antimicrobial surfactant improved the antimicrobial activity of the fabric, as demonstrated by antimicrobial test methods for antibacterial activity assessment. Using different, complementary antimicrobial compounds mixed together and applied as a coating, it resulted of a greater zone of inhibition evaluated with the parallel streak test method AATCC 147, while the biocidal fabric maintained a high level of performance after commercial washing or autoclaving.

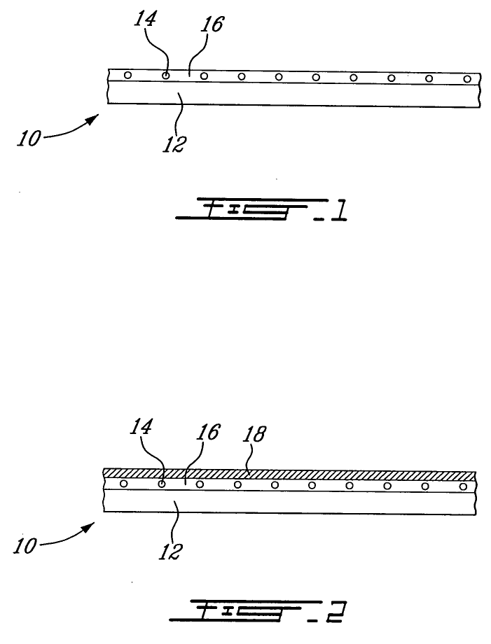

As illustrated in FIG. 1, the material (10) of a preferred embodiment of the present invention consists in a sheet of fabric (12) covered with metallic particles (14), such as metallic salt crystals, preferably silver chloride, which are embedded in an adhesive (16). The adhesive (16) offers the advantage to promote slower release of the metallic particles (14) and therefore provides an improved antimicrobial activity.

FIG. 2 illustrates an embodiment of the material (10) wherein a layer of metal (18), consisting in a layer of silver having been deposited by plasma, covers the metallic particles (14) embedded in the adhesive (16).

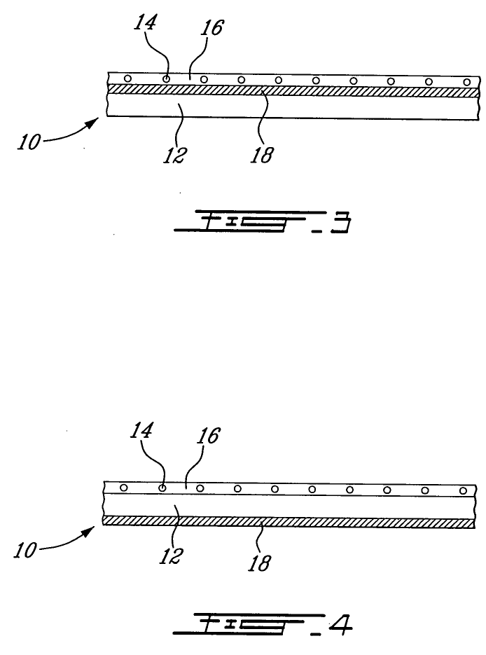

FIG. 3 illustrates an embodiment of the material (10) wherein the layer of metal (18) is between the metallic particles (14) embedded in the adhesive (16) and the sheet of fabric (12).

FIG. 4 illustrates an embodiment of the material (10) wherein the layer of metal (18) is underneath the sheet of fabric (12).

When producing the embodiment illustrated in FIG. 1, the fabric is passed through a first bath of dissolved silver salt in concentration ranging from 0.001 M to 1.0 M, more preferably, from 0.1 to 0.3 M. The silver salt may be silver bromide, silver perchlorate, silver fluoride, silver chloride, silver nitrate, silver sulfate, silver iodide, silver alkylcarboxylate, silver sulphadiazine, silver arylsulfonate, or other soluble silver salt.

Then, the fabric is passed through a second bath into which is dissolved a second salt slightly in excess with the former. This salt may be either dissolved in pure water or water/alcoholic compound/acetone mixture. The alcoholic compound should preferably be a non-leachable ethoxylated alcohol or a mixture of alcoholic compounds.

This second salt is formed of an anion which possess the ability of coupling with the silver cation then forming a salt colloid that can precipitate and deposit onto the surface of the fabric. The precipitation of the silver salt is achieved by variation in water/alcoholic compound/acetone mixture, temperature or addition of an antimicrobial surfactant or a combination thereof. The silver salt thus precipitated is slightly soluble and allows to achieve controlled-release of antimicrobial silver species over time. To this second bath may be added an antimicrobial and surfactant compound, such as Quaternary Ammonium Compounds (QAC), chlorinated organic compounds, alcohols, or others antimicrobial substances to improve the stability and the antimicrobial properties of the colloidal solution.

It is well-known from the art that silver nitrate or any other water soluble silver salt possesses a lower solubility in alcohol. Provided that the solubility of the former salt may vary upon the relative concentration of the alcohol, controlled precipitation of the resulting silver salt colloid using the water/alcoholic compound/acetone mixture in the second bath may be achieved.

To a third bath is added an monomer emulsion composed of a monomer having film forming and binding capabilities with the fabric and the silver salt precipitated onto the fabric. Different families of monomers may be used to form the adhesive coating to the fabric, namely acetates, acrylates, acrylics, acrylamides, urethanes, vinyls, esters, and co-monomers thereof in a manner to obtain either polymers or co-polymers. More preferably, polymers or co-polymers depicting good adhesion or binding affinities to the fabric's fiber will be chosen.

The precipitated silver salt is present under the form of colloids which are embedded into a polymer or co-polymer top layer thermally bonded to the fabric base layer. The thermal bonding is achieved at a suitable drying/curing temperature for the top and base layers, respectively. The silver salt adhesive layer may be applied either one or both sides of the base fabric layer.

As illustrated in FIG. 2, the material (10) can be silver sputtered using a plasma process. The deposited silver layer will have a thickness ranging from several tenth of nanometers to one micron. Processing gas for sputter-deposition of silver can be either pure argon or argon/oxygen blend of gas to form respectively a nanocrystaline silver or silver oxide coating having anti-microbial properties. Other processing gas can be selected as described earlier. The same process is used to prepare the embodiments described in FIGS. 3 and 4, except that for the embodiment described in FIG. 3, the silver sputtering step has to be performed before passing the fabric into the baths to form the coat of silver salt crystals embedded in adhesive.

Other antimicrobial compounds may be used as described below. Among them is an antimicrobial, chlorinated, organic compound known as Chlorinated organic, non-leaching, antimicrobials agents of this type may be chosen from the group consisting of 2,4,4′-trichloro-2′-hydroxy diphenol ether and 5-chloro-2-phenol (2,4-dichlorophenoxy). Another antimicrobial compound is chlorhexidine, which is active against gram-positive and gram-negative organisms, facultative anaerobes, aerobes, and yeast. Chlorhexidine is also known as Chlorhexidine Base or 5,5′-bis(4-chlorophenyl)-1,1′-hexamethylenedibiguanide. Other possible antimicrobial agents are Cetrimide, Hexachlorophene, Iodine Compounds, Alcoholic compounds, Hexamine Hippurate, and Dequalinium.

Evaluation of Properties

The antimicrobial fabric may be rendered electrically conductive through the deposition of a silver coating or the incorporation of a carbon yarn in the fabric pattern. The suitable surface resistivity level would be, more prefer, lower than 1 Ohm/square, when measured with a method like the one described in MTCC Test Method 76-1995 [symbol] Electrical Resistivity of Fabrics [symbol] .

The adhesion of the silver or silver salt coated layer to the fabric can be measured, dry and wet, using a practical test method CAN/CGSB 4.2 NO. 22-M90 entitled [symbol] Colorfastness to Crocking [symbol] . This method provide qualitative ratings indicating the adhesion quality of the coated layer to the fabric base layer. The appreciation for the performance of colorfastness to crocking is given from number 5 to 1. On this scale, 5 is excellent colorfastness and 1 is very poor colorfastness to crocking.

Kill rate performance was evaluated using the Dow Corning Corporate Test Method CTM 0923 [symbol] Antimicrobial Activity—Dynamic Test of Surfaces [symbol] . The tested bacteria was P. Aeruginosa. The antimicrobial-treated fabric sample and a control fabric were separately put in contact with the bacteria media for 2 hours, and the count at the beginning and after 2 hours is noted. After calculations, the result is expressed in term of percent reduction (%) of the bacteria.

Antimicrobial activity assessment of the fabric. The antimicrobial activity was assessed according the AATCC Test Method 147-1998 which is described here and which is a qualitative procedure that demonstrates the bacteriostatic activity by the diffusion of the antibacterial agent through agar. Following is the procedure for the evaluation: examine the incubated plates for interruption of growth along the streaks of inoculums beneath the specimen and for a clear zone of inhibition beyond its edge. The average width of a zone of inhibition along a streak on either side of the test specimen may be calculated using the following equation:
W=(T−D)/2

Where:

W=width of clear zone of inhibition in mm

T=total diameter of test specimen and clear zone in mm

D=diameter of the test specimen in mm

An alternative method for the evaluation of the inhibition zone consists in incubating the sample as previously described, the recto side (as illustrated in FIG. 10) against the culture, at a temperature of 37° C. during 24 hours on Mueller-Hinton agar plates. The length of the inhibition zone is determined by measuring the length of the inhibition zone at the periphery of the 2 longer sides and calculating the mean value.

### EXAMPLE 1

A knitted polyester/carbon fabric (90/10) is passed through a bath containing a silver nitrate solution 0.2 M and then in a second bath containing a vinyl acetate emulsion into which dissolved sodium chloride is in excess 0.25 M which allows the formation of silver chloride colloid of a size range of 10-1000 nm. The silver chloride colloid dispersed into the vinyl acetate emulsion is subsequently squeezed inside the fabric using rubber laminated rolls before being dried on a finishing line to the temperature of 150° C. at a speed of 0.3 metre/minute. An electrically conductive, crocking resistant, antimicrobial fabric possessing antibacterial properties against gram negative and gram positive bacteria is obtained.

Surface resistivity, Ohm/square: <1

Crocking dry, 5-1: 4-5

Crocking humid, 5-1: 4

Inhibition, S. Aureus (presence or absence): presence

Inhibition, P. Aeruginosa (presence or absence): presence

Inhibition, E. Faecium (presence or absence): presence

Inhibition Zone, S. Aureus (mm): <1

Inhibition Zone, P. Aeruginosa (mm): <1

Inhibition Zone, E. Faecium (mm): 2

Kill rate, 2 hours, (%): 100

### EXAMPLE 2

A knitted polyester/carbon fabric (90/10) is passed through a bath containing a silver nitrate solution 0.2 M and then in a second bath containing a solvent mix prepared in equal parts of water and ethanol (50:50%/vol) into which is dissolved sodium chloride is in excess 0.25 M which allows the formation of silver chloride colloid of a size range of 10-1000 nm. Also to this second bath is added a chlorinated organic compound, in the occurrence an organic compound known as Triclosan®. Chlorinated organic, non-leaching, antimicrobials agents of this type may be chosen from the group consisting of 2,4,4′-trichloro-2′-hydroxy diphenol ether and 5-chloro-2-phenol (2,4-dichlorophenoxy). The third bath contains a vinyl acetate emulsion. The silver chloride colloid dispersed into the vinyl acetate emulsion is subsequently squeezed inside the fabric using rubber laminated rolls before being dried on a finishing line to the temperature of 150° C. at a speed of 0.3 metre/minute. By admixing an antimicrobial surfactant, in occurrences Triclosan®, an electrically conductive, crocking resistant, antimicrobial fabric possessing improved antibacterial properties against gram negative and gram positive bacteria is obtained.

Surface resistivity, Ohm/square: <1

Crocking dry, 5-1: 4-5

Crocking humid, 5-1: 4

Inhibition, S. Aureus (presence or absence): presence

Inhibition, P. Aeruginosa (presence or absence): presence

Inhibition, E. Faecium (presence or absence): presence

Inhibition Zone, S. Aureus (mm): 5

Inhibition Zone, P. Aeruginosa (mm): <1

Inhibition Zone, E. Faecium (mm): 2

Kill rate, 2 hours, (%): 100

### EXAMPLE 3

A woven nylon fabric is passed through a bath containing a silver nitrate solution 0.2 M and then in a second bath containing 0.25 M sodium chloride solution which allows the formation of silver chloride colloids of a size range of 10-1000 nm. Also to this second bath is added a chlorinated organic compound, in the occurrence an organic compound known as Triclosan®. Chlorinated organic, non-leaching, antimicrobials agents of this type may be chosen from the group consisting of 2,4,4′-trichloro-2′-hydroxy diphenol ether and 5-chloro-2-phenol (2,4-dichlorophenoxy). The third bath contains a polyurethane emulsion. The silver chloride colloid dispersed into the polyurethane emulsion is subsequently squeezed inside the fabric using rubber laminated rolls before being dried on a finishing line to the temperature of 150° C. at a speed of 0.3 metre/minute. An electrically conductive, crocking resistant, antimicrobial fabric possessing domestic wash durability is obtained. Washing and drying cycles are performed according to standard test method ISO 6330/675.

Surface resistivity, Ohm/square: <1

Crocking dry, 5-1: 4-5

Crocking humid, 5-1: 4

Inhibition, S. Aureus (presence or absence): presence

Inhibition, P. Aeruginosa (presence or absence): presence

Inhibition, E. Faecium (presence or absence): presence

Initial

Inhibition Zone, S. Aureus (mm): 2

Inhibition Zone, P. Aeruginosa (mm): 1

Inhibition Zone, E. Faecium (mm): 2

After One Washing and Drying Cycles

Inhibition Zone, S. Aureus (mm): 1

Inhibition Zone, P. Aeruginosa (mm): <1

Inhibition Zone, E. Faecium (mm): 2

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An antimicrobial material comprising
a sheet of fabric and;
metallic particles embedded in an adhesive material covering said sheet of fabric.

2. The material of claim 1, wherein said particles are selected from the group consisting of salt crystals, oxide particles and hydroxide particles.

3. The material of claim 2, wherein said crystals are micro-sized crystals.

4. The material of claim 2, wherein said crystals are nano-sized crystals.

5. The material of claim 2, wherein said crystals are in a size range from 10 to 1000 nm.

6. The material of claim 2, wherein said crystals are in a size range from 10 to 500 nm.

7. The material of claim 2, wherein said crystals are in a size range from 10 to 150 nm.

8. The material of claim 1, wherein said fabric is selected from the group consisting of nylon, aramid, acetate, flax, polyolefin such as polyethylene and polypropylene, polyester, rubber, saran, spandex, vinyl, vinyon, cotton, wool, silk, rayon, glasswool, acrylic, paper, polytetrafluoroethylene, synthetic polymers, cellullosic fibers, natural fibers, synthetic or man made fibers and mixtures thereof.

9. The material of claim 1, wherein said fabric is nylon.

10. The material of claim 1, wherein said fabric is polyester/carbon.

11. The material of claim 1, wherein said fabric is polyester/cotton.

12. The material of claim 1, wherein said fabric is a membrane made of a material selected from the group consisting of polytetrafluoroethylene, polyurethane, polyester and copolymers.

13. The material of claim 1, wherein said fabric is electrically conductive.

14. The material of claim 13, wherein said fabric is rendered electrically conductive by the incorporation of electricity conductive material thereto.

15. The material of claim 13, wherein said electricity conductive material is selected from the group consisting of metallic yarn, carbon yarn and a combination thereof.

16. The material of claim 2, wherein said metallic salt crystals are from a metal selected from the group consisting of silver, platinum, gold, copper, zinc, titanium, magnesium and mixtures thereof.

17. The material of claim 2, wherein said metallic salt crystals are selected from the group consisting of AgBr, silver perchlorate, AgF, AgCl, AgNO₃, silver sulfate, AgI, silver alkylcarboxylate, silver sulphadiazine, silver arylsulfonate and mixtures thereof.

18. The material of claim 2, wherein said metallic salt crystals are silver chloride crystals.

19. The material of claim 2, wherein said metallic salt crystals are selected from the group consisting of CuI, CuBr, CuCl, CuF, CuBr₂, CuCl₂, CuI₂, and CuF₂

20. The material of claim 2, wherein said metallic salt crystals are selected from the group consisting of AuF₃, AuCl, AuCl₃, AuBr₃ and AuI.

21. The material of claim 2, wherein said metallic oxides are from a metal selected from the group consisting of silver, platinum, gold, copper, zinc, titanium, magnesium and mixtures thereof.

22. The material of claim 2, wherein said metallic oxides are selected from the group consisting of Ag₂O and AgO.

23. The material of claim 2, wherein said metallic oxides are selected from the group consisting of Cu₂O and CuO.

24. The material of claim 2, wherein said metallic oxides are Au₂O₃.

25. The material of claim 2, wherein said metallic hydroxides are from a metal selected from the group consisting of silver, platinum, gold, copper, zinc, titanium, magnesium and mixtures thereof.

26. The material of claim 2, wherein said metallic hydroxides are Ag(OH).

27. The material of claim 2, wherein said metallic hydroxides are Cu(OH)₂.

28. The material of claim 1, further comprising metallic silver embedded in said adhesive material.

29. The material of claim 1, further comprising metallic copper embedded in said adhesive material.

30. The material of claim 1, further comprising an antimicrobial compound.

31. The material of claim 30, wherein said antimicrobial compound is selected from the group consisting of quaternary ammonium compounds (QAC), chlorinated organic compounds, cetrimide, iodine compounds, hexamine hippurate, dequalinium and alcohols.

32. The material of claim 31, wherein said chlorinated organic compound is selected from the group consisting of 2,4,4′-trichloro-2′-hydroxydiphenol ether, chlorhexidine, hexachlorophene and 5-chloro-2-phenol (2,4-dichlorophenoxy).

33. The material of claim 1, wherein said adhesive is made of monomers selected from the group consisting of acetate, acrylate, acrylic, acrylamide, urethane, vinyl, ester and antimicrobial polymer.

34. The material of claim 33, wherein said antimicrobial polymer is selected from the group consisting of siloxane polymer having been functionalized by N-halamines, iodinated resin, iodinated complex, polymeric biguanide compound and related cationic salt derivatives, polymerized aromatic quaternary ammonium salt monomers, poly(2-propenal, 2-propenoic acid), α,β-amino acid oligomer or polymer, and poly(2-methyl-5-vinylpyridine) or poly vinylpyrrolidone treated by iodide salt.

35. The material of claim 34, wherein said polymeric biguanide compound is poly(hexamethylene biguanide).

36. The material of claim 1, wherein said adhesive is vinyl acetate.

37. The material of claim 1, wherein said adhesive is polyurethane.

38. The material of claim 1, further comprising a layer of metal over said metallic ions embedded in adhesive material.

39. The material of claim 1, further comprising a layer of metal between said sheet of fabric and said metallic ions embedded in adhesive material.

40. The material of claim 1, further comprising a layer of metal underneath said sheet of fabric.

41. The material of any one of claims 38 to 40, wherein said metal is silver.

42. The material of claim 41, wherein said silver is nanocrystalline silver.

43. The material of claim 41, wherein said silver is silver oxide.

44. The material of any one of claims 38 to 43, wherein said layer of metal is formed by plasma deposition.

45. The material of claim 44, wherein said plasma used for deposition is selected from the group consisting of argon, argon/oxygen, argon/nitrogen, argon/nitrogen/hydrogen, krypton, krypton nitrogen, krypton/nitrogen/hydrogen, xenon, xenon/nitrogen, xenon/nitrogen/hydrogen, helium, helium/nitrogen, helium/nitrogen/hydrogen, neon, neon/nitrogen and neon/nitrogen/hydrogen plasma.

46. The material of claim 44, wherein said plasma used for deposition is argon plasma.

47. The material of claim 1, wherein said material affects gram positive and gram negative bacteria.

48. The material of claim 1, wherein said gram positive bacteria are selected from the group consisting of staphylococcus aureus and bacillus anthracis.

49. The material of claim 1, wherein said gram negative bacteria are selected from the group consisting of Escherichia coli, pseudomonas aeruginosa, enterococcus faecium and salmonella.

50. The material of claim 1, wherein said material have an antiviral activity.

51. The material of claim 1, wherein said material have an antifungal activity.

52. The material of claim 1, wherein said adhesive material is transparent.

53. The material of claim 1, wherein said adhesive material is washing durable.

54. The material of claim 1, wherein said fabric is antistatic.
